(19) United States
(12) Patent Application Publication
(10) Pub. No.: US 2025/0295509 A1
(43) Pub. Date: Sep. 25, 2025

(54) VASCULAR STENT DEPLOYMENT SYSTEM

(71) Applicants: Stanley Batiste, Granite Bay, CA (US); Daniel Batiste, Roseville, CA (US)

(72) Inventors: Stanley Batiste, Granite Bay, CA (US); Daniel Batiste, Roseville, CA (US)

(21) Appl. No.: 19/088,244

(22) Filed: Mar. 24, 2025

Related U.S. Application Data
(60) Provisional application No. 63/569,427, filed on Mar. 25, 2024.
(60) Provisional application No. 63/651,885, filed on May 24, 2024.

(51) Int. Cl. A61F 2/966

(52) U.S. Cl. CPC A61F 2/966; A61F 2002/9665

(57) ABSTRACT

A vascular stent deployment system is delivered to a repair site in the body by means of a catheter. The deployment system apparatus comprises a vascular stent to effect the repair, a delivery sheath in which the unexpanded vascular stent is delivered to the repair site, and a pusher rod for ejecting the stent from the delivery sheath for deployment. The system further comprises a stent assist wire, located in relation to the stent, which is positioned in relation to a known feature of the vasculature, thereby assuring that the stent is accurately deployed at the repair site.

### CROSS-REFERENCE TO RELATED APPLICATIONS

This application claims benefit under Title 35, United States Code § 119(e) of U.S. Provisional Application No. 63/569,427 filed on Mar. 25, 2024 and U.S. Provisional Application No. 63/651,885 filed on May 24, 2024.

### FIELD OF THE INVENTION

This invention relates to vascular stents, and in particular to systems for precise deployment of vascular stents by minimally invasive procedures.

### BACKGROUND OF THE INVENTION

Vascular stents were developed in the 1980s and started to gain widespread acceptance in the 1990s following FDA approval for the repair and remediation of serious vascular conditions such as coronary heart disease, peripheral artery disease, and aneurysms. Stents are fabricated as tubular mesh structures made of metal, polymer or fabric, and may include a polymer coating designed for elution of anti-fibrotic drugs and retarding encapsulation. The stents can be either covered (commonly using PTFE) or uncovered, the latter generally referred to as “bare metal” stents. The mesh structure is collapsible to a small cylindrical structure for intravenous catheter delivery to the site of the affliction, and can thereafter be expanded to exert radial pressure in a vessel, either automatically by spring-like construction or by inflation of a balloon catheter inside the lumen of the stent. The radial pressure of an expanded stent maintains the stent in its implant location where it will compress plaque obstruction of the vessel or support and strengthen the wall of the vessel at the location of a developing aneurysm.

An attractive feature of vascular stents is that they can be implanted by a minimally invasive surgical procedure using a catheter that is threaded through the vascular system to the site requiring repair. The minimal bodily stress imposed by such procedures enables most patients to recover quickly and resume normal lifestyles. Catheter delivery is made possible by the use of real-time x-ray imaging (fluoroscopy), enabling the clinician to guide the catheter to the site in the vasculature where the stent must be delivered and implanted. This procedure, however, requires a great deal of skill and expertise, as the image guidance provided by fluoroscopy is minimal. Since fluoroscopy is an x-ray modality, it cannot provide distinct images of soft tissue structures like blood vessels. Consequently the clinician is mainly observing bones and the catheterization apparatus, the latter usually bearing radiopaque markings, and is relying largely on prior experience and educated estimation while trying to deploy the stent at the exact location in the vasculature where it is needed. Further complicating the procedure is the fact that the insertion and manipulation of the catheter apparatus can distort and distend the tissues at an around the site of the procedure. The clinician will usually plan the procedure with an angiogram of the site, with vessel lumens and the site requiring repair distinctly identified by the presence of a contrast medium. Such advance mapping of the procedure is of minimal assistance when the anatomy is distorted during the procedure to different dimensions and locations than appeared in the preparatory angiogram.

As a result, the stent may ultimately be deployed in a slightly different location or orientation than that which was intended. This can be particularly problematic when the implant site is at or near the confluence of several blood vessels. An example of such a problematic deployment is illustrated in FIG. 1. In this case it was intended to deploy the stent 150 at the terminus of the left common iliac vein 12, just below its jointure with the contralateral common iliac vein 11 and the inferior vena cava 10. However, as the drawing illustrates, the stent was mispositioned such that it projects into the inferior vena cava 10 and also crosses the flow pathway of the right common iliac vein 11, where it can impede the venous flow and possibly cause right iliac vein thrombosis. Stents that extend into other flow pathways, termed “jailing” by interventional radiologists and vascular surgeons, can impede flow of the crossed pathway. Bare metal stents will allow blood to traverse the stent interstices which in time can become occluded by fibrin buildup, further increasing the resistance to flow. Eventually the flow pathway will be fully impeded and the jailed vessel will occlude. If this occurs in the iliac veins' confluence it will result in contralateral deep vein thrombosis.

Stents will also exhibit effects of aging in the body. Stent fracture is a recognized complication of intravascular stent implantation, with the primary cause being mechanical stress where repetitive contractions expose stents to forces such as compression, torsion, kinking, elongation, bending, and shear stress. These forces can lead to mechanical fatigue and eventual fracture of the stent material. Stent fractures can lead to a range of clinical complications, depending on their severity and location. Fractured stents can trigger thrombosis due to abnormal endothelialization and local mechanical irritation. This can result in occlusion of the stent which, in the case where the stent crosses into the contralateral flow pathway, causes stenosis or occlusion of flow. Ultimately this problem can result in contralateral venous thrombosis and, if in the pelvis, lower extremity deep vein thrombosis.

### SUMMARY OF THE INVENTION

Accordingly it is an object of the present invention to enable precise catheter deployment of stents within blood vessels, preferably to millimeter accuracies. It is a further object of the present invention to provide such accuracy when a stent is to be implanted at or near the location of a confluence of blood vessels in the body.

In accordance with the principals of the present invention, the catheter-borne deployment apparatus for a vascular stent comprises a stent assist wire which assures that a stent is deployed with precision in relation to a known feature of the vasculature. In a preferred implementation the known feature is the confluence of several vessels in the body. The stent assist wire is attached to the stent or to the stent deployment apparatus. The stent assist wire is positioned in or around the known feature, enabling the deployment to occur with precision in relation to the known feature of the vasculature.

In a preferred implementation the catheter-borne deployment apparatus for a vascular stent comprises a vascular stent; a delivery sheath; a pusher rod; and a stent assist wire, located in relation to the stent, and extending outward from the vascular stent or deployment apparatus, the stent assist wire adapted to be positioned in relation to a known feature of the vasculature, wherein the vascular stent is adapted to be deployed when the stent assist wire is positioned in relation to the known feature.

### OBJECTS OF THE INVENTION

Accordingly, a primary object of the present invention is to provide a stent assist wire, which assists in placement of a stent at a desired location within a patient's vasculature.

Another object of the present invention is to provide a stent assist wire which can be positioned precisely relative to a patient's vasculature and in turn assist positioning of a stent where desired.

Another object of the present invention is to provide a stent assist wire which can be transitioned between a collapsed form into a deployed form to facilitate easier routing through the vasculature to a stent implantation site and be deployed into a larger form where desired.

Another object of the present invention is to provide a stent which is configured to simplify precise stent placement where desired, and improve probability that a stent will be placed precisely where desired.

Another object of the present invention is to provide a deployment system and method for a stent which system and method simplify precise stent placement and improve a probability that a stent will be placed precisely where desired.

Other further objects of the present invention will become apparent from a careful reading of the included drawing figures, the claims and detailed description of the invention.

### BRIEF DESCRIPTION OF THE DRAWINGS

FIG. 1 is an illustration of an inaccurately deployed stent obstructing the flow at a confluence of blood vessels.

FIG. 2 illustrates an angiogram of the common iliac veins and their confluence with the inferior vena cava.

FIG. 3 illustrates a stent with an attached stent assist wire in accordance with the present invention.

FIG. 4 illustrates the stent and stent assist wire of FIG. 3 when packaged for catheter deployment.

FIG. 5 illustrates the stent and stent assist wire of FIG. 4 when packaged for delivery in a deployment sheath with a pusher rod.

FIGS. 6 through 11 is a sequence of illustrations of the deployment of the stent with the attached stent assist wire of FIG. 3.

FIGS. 12 through 14 is a sequence of illustrations of the deployment of a stent a precise distance away from the confluence of two blood vessels.

FIG. 15 illustrates a stent packaged for delivery with a partially opened stent assist wire.

FIGS. 16 through 18 illustrate the deployment of the packaged stent and stent assist wire of FIG. 15.

FIG. 19 illustrates the packaging of a stent of the present invention with a vascular wire delivered in a wire guide catheter that serves as the stent assist wire.

FIGS. 20 through 27 illustrate the delivery of a catheter by the stent delivery system of FIG. 19.

FIG. 28 illustrates a stent delivery system of the present invention with a stent assist wire extending from the trailing end of the stent.

FIGS. 29 through 34 are a series of drawings illustrating the deployment of the stent delivery system of FIG. 28 at an orthogonal confluence of blood vessels.

FIGS. 35 and 36 illustrate a variation of the stent delivery system of FIG. 28 in which the stent assist wire is attached to the stent pusher rod and removed from the body with the withdrawal of the catheter delivery system.

FIGS. 37 to 42 illustrate a variation in configuration of FIG. 19 where a vascular wire is delivered in a wire guide catheter that serves as the stent assist wire configured with an extended tip design.

FIGS. 43 and 44 illustrate the deployment of a stent with an extended tip design in accordance with the principles of the present invention.

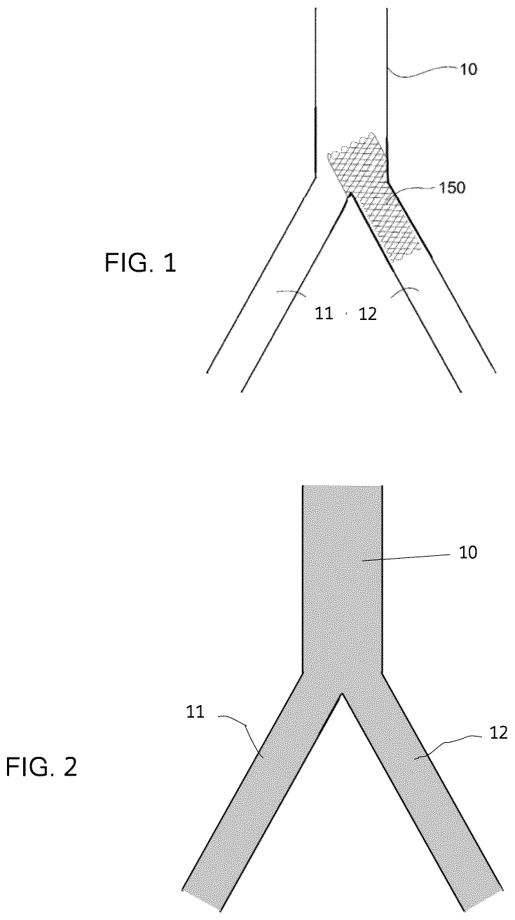

### DESCRIPTION OF THE PREFERRED EMBODIMENT

A vascular stent is preferably implanted in accordance with the present invention by an image-guided catheter-borne procedure. Prior to the procedure, an angiogram is acquired by fluoroscopy, using contrast media which distinctly depicts blood flow and flow abnormalities in the vessels of interest. The width of the blood in a lumen will appear narrowed where plaque has built up on a vessel wall, for instance. FIG. 2 depicts an angiogram of the oblique confluence of the right and left common iliac veins 11 and 12 with the inferior vena cava 10. The gray shading in the vessels illustrates how distinctly the contrast-enhanced blood flow of the vessels stands out against the surrounding area. The angiogram is used by a clinician to plan the deployment of a stent, mapping the exact location where a stent should be implanted. During the deployment procedure, a digital form of the angiogram can be overlaid over the real-time fluoroscopic image or displayed alongside the real-time image to help guide the placement of the stent.

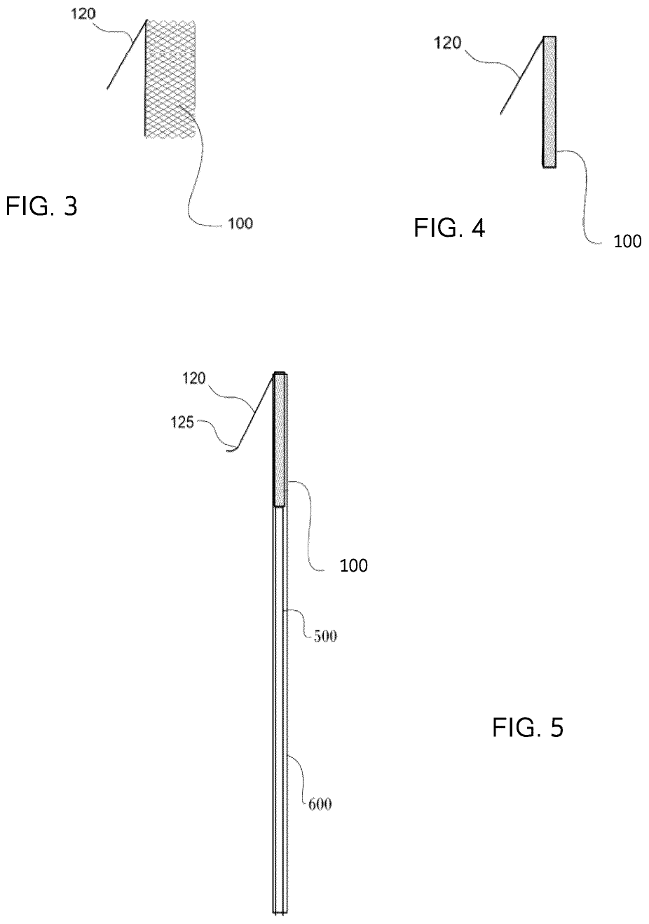

But as previously mentioned, the forces applied in the body at the site of a procedure by the motion and manipulation of the catheter-borne instrumentation can distort and distend the local anatomy and its vasculature, so that the anatomy at the time of the procedure will not exactly match the anatomical map of the preparatory angiogram. This problem is overcome by an implementation of the present invention such as the stent 100 shown in FIG. 3.

In this illustration the stent is shown in its fully expanded form as it appears when deployed in a blood vessel. In the implementation of FIG. 3 the stent 100 has a stent assist wire 120 attached to a side of the stent. The stent assist wire in this embodiment comprises a vertical section (also called a base section) affixed to the side of the stent (in this embodiment), and an angled section which extends outward from the vertical section and is joined to the base section at an apex. This apex has an angle of less than 180 degrees when in a relaxed and unloaded state. The apex can be loaded by forces to collapse the apex, such as to make the stent assist wire narrower during placement. In one embodiment, a removable sheath provides these forces. When forces are removed, the apex returns to its unloaded angle. This angle can be obtuse, acute or a right angle.

The stent assist wire may be made of, for instance, stainless steel, nickel-titanium (nitinol), cobalt-chrome alloys, tantalum and tungsten which can be coated with materials such as PTFE, polyurethane and other hydrophilic polymer coatings. The stent assist wire may also made of materials the dissolve or reabsorb in the vascular system such as polymers like Poly(L-lactide) (PLLA), Poly(lactide-co-glycolic acid), Poly glycolic acid and Poly-ε-caprolactone. Metals such as Magnesium and its alloys, Zinc and its alloy and Iron as well as other materials such as Tyrosine poly carbonate, Salicylic acid2 and Polydiolcitrate: A light-curable material incorporating methacrylate groups. The stent assist wire may be formed as an integral part of the stent during the stent manufacturing process, or it may be formed separately and attached to the stent by spot welding, soldering, or being crimped onto the stent. It may also be adhesively attached to the stent. FIG. 4 shows the stent and stent assist wire with the stent in its fully collapsed form for insertion into a catheter-borne delivery sheath 600 as shown in FIG. 5. The collapsed stent is seen to be located at the upper end of the sheath, with the angled section 120 of the stent assist wire located outside of the sheath. In this implementation the angled section 120 terminates in a curved distal tip 125 which protects the vessel during stent deployment. The stent 100 is mounted on a pusher rod 500 within the sheath 600, which is advanced to eject the stent from the sheath for stent deployment.

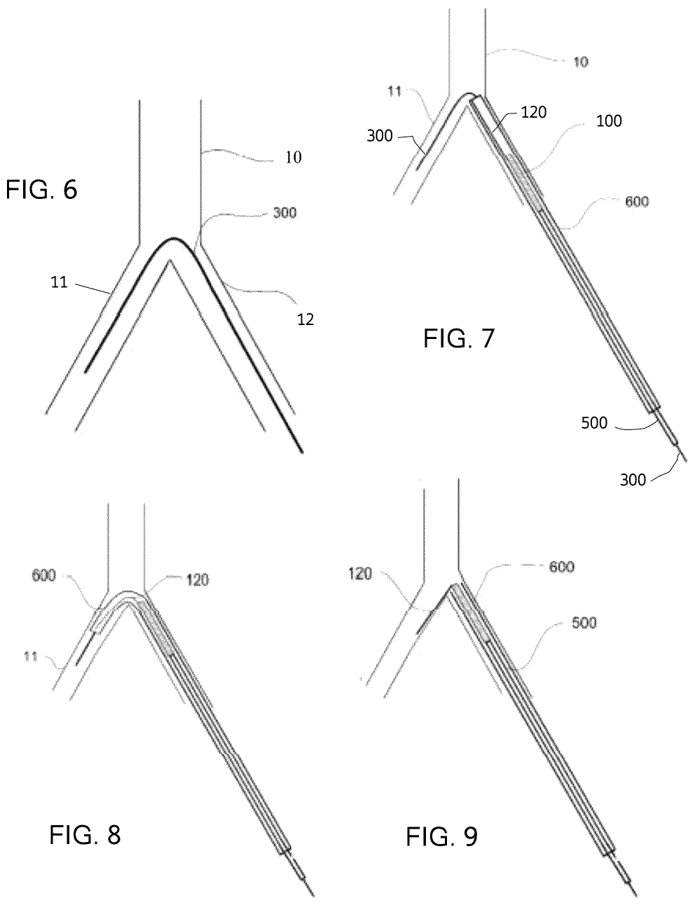

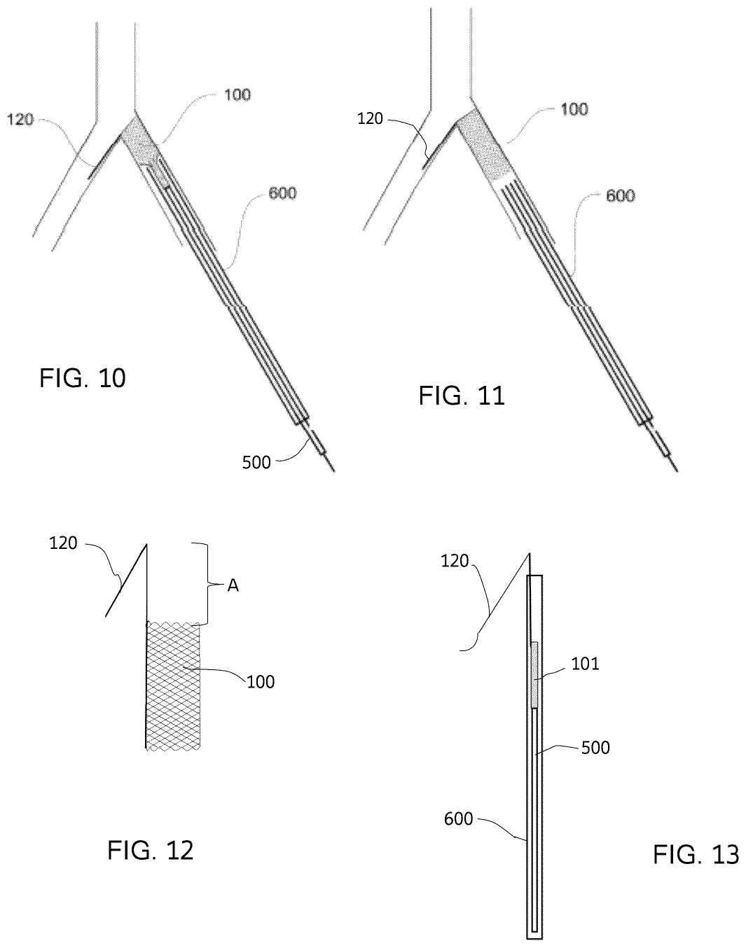

The positioning of the stent within a blood vessel and its deployment at an intended site within the vessel is illustrates in the sequence of drawings of FIGS. 6 through 11. In FIG. 6 a guide wire 300 is first advanced through the common iliac vein 12 and crosses through its confluence with the inferior vena cava 10 and is further advanced into the contralateral common iliac vein 11. FIG. 7 then shows the advancement of the delivery system of FIG. 5 over the guide wire 300. In this particular implementation the stent is not located at the very top of the deployment sheath 600, but in a retracted position with the stent assist wire 120 extending forward of the stent inside the sheath. In FIG. 8 the delivery system has been further advanced until the extended stent assist wire 120 is located in the contralateral common iliac vein 11. The stent is now positioned with its leading edge at the confluence of the iliac veins. The deployment sheath 600 is then pulled back while holding the pusher rod 500 in a stationary position, uncovering the stent assist wire 120 which is in the contralateral vessel. The deployment system is pulled back further, snugging the stent assist wire 120 against the medial wall of the contralateral iliac vein as shown in FIG. 9. With the stent assist wire now firmly positioning the tip of the stent at the confluence of the common iliac veins, the deployment sheath is pulled back further while maintaining the position of the pusher rod 500 which begins to eject the stent from the deployment sheath as shown in FIG. 10. The self-expanding stent 100 begins to expand as shown in FIG. 10 and is shown fully deployed in FIG. 11. The deployment system including the guide wire 300, the pusher rod 500, and the deployment sheath 600 is then withdrawn from the body.

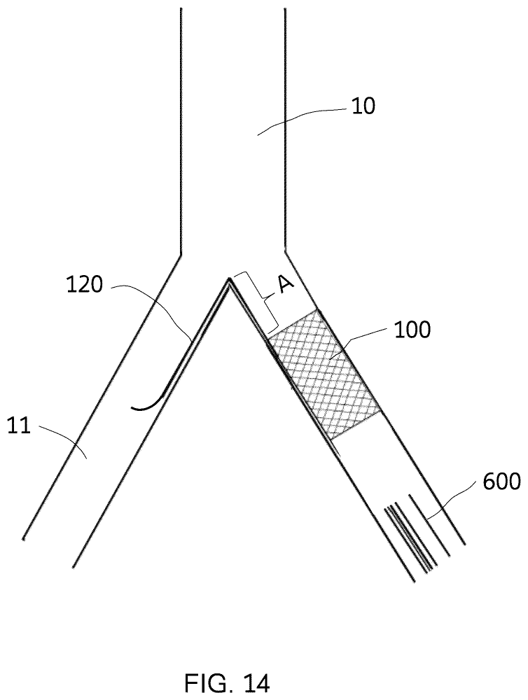

FIGS. 12 through 14 illustrate the structure and deployment of a stent 100 which again has a stent assist wire 120 attached to the side of the stent as in FIG. 3, but in this case the stent assist wire is configured so as to deploy the stent at a precisely determined location below the confluence of blood vessels. In this example the section of the stent assist wire 120 which is attached to the side of the stent extends above the stent 100 for a distance “A” before the angled section extends outwardly. This enables the stent to be positioned with its center located a predetermined distance below a vessel confluence. For example, suppose the stent has a length of 12 mm, and it is desired to deploy the stent with its center over a vessel defect which is located 10 mm below the confluence of the common iliac veins. In that case, half of the stent length is 6 mm and, if dimension A is set to be 4 mm, then the stent will be precisely deployed with its center over the vessel defect. The stent of FIG. 12 may be provided with a straight stent assist wire extending from the stent, which is then creased by the clinician exactly 4 mm above the end of the stent to provide dimension A from which the angled section extends. Alternatively, the stent may be manufactured with the stent assist wire accurately dimensioned as shown in FIG. 12 for the procedure. FIG. 13 shows the collapsed stent 101 and its pusher rod 500 positioned in the delivery sheath 600 and the stent assist wire 120 extending out the end of and away from the sheath. When the stent is advanced so that the stent assist wire 120 with its angled bend is positioned at the confluence of the common iliac veins as shown in FIG. 14, it is seen that the center of the deployed 12 mm stent 100 is precisely positioned 10 mm below the confluence of the common iliac veins (A=4 mm plus half of the stent length=6 mm). The deployment sheath 600 and the rest of the deployment apparatus is then withdrawn from the site of the deployed stent.

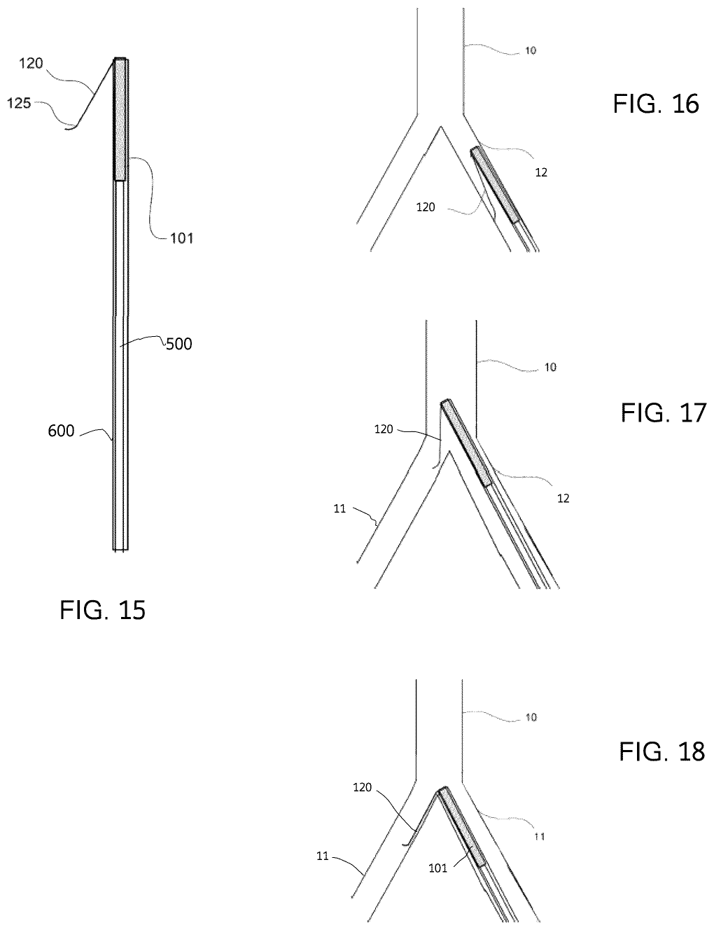

FIGS. 15 through 18 illustrate another implementation of the present invention in which the angled section 120 of the stent assist wire is located outside of the deployment sheath during the entirety of deployment. The stent assist wire 120 may take one of three configurations in this example. It may be attached to the stent as in the implementation of FIG. 3; it may be attached to the deployment sheath 600; or it may comprise a guide wire not attached to the deployment system and extending the full length of the catheter. In the first case the stent assist wire will remain in the body; in the latter cases the stent assist wire will be removed from the body with the withdrawal of the stent deployment system from the body. In FIG. 15 a stent 101 is mounted on pusher rod 500 and enclosed in deployment sheath 600. The angled section of the stent assist wire 120 has a curved distal tip 125 to protect the vessel during deployment. In FIG. 16 the delivery system is seen advancing inside of the common iliac vein 12 and the stent assist wire 120 is continuously open and engaging the medial wall of the vein 12. In FIG. 17 the system is shown further advanced into the inferior vena cava 10 with the stent assist wire 120 extending into the contralateral iliac vein 11. The deployment sheath and pusher rod are then pulled back as shown in FIG. 18, seating the stent assist wire 120 against the medial wall of the iliac vein. The stent is then deployed at the top of the left common iliac vein 11 where it is positioned as shown in FIG. 18.

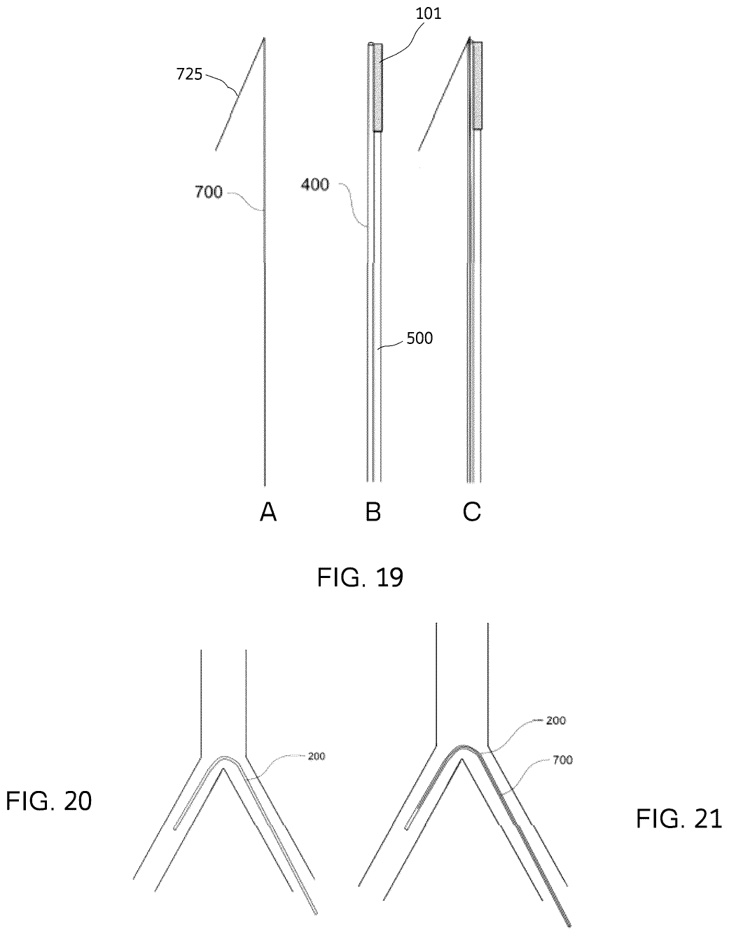

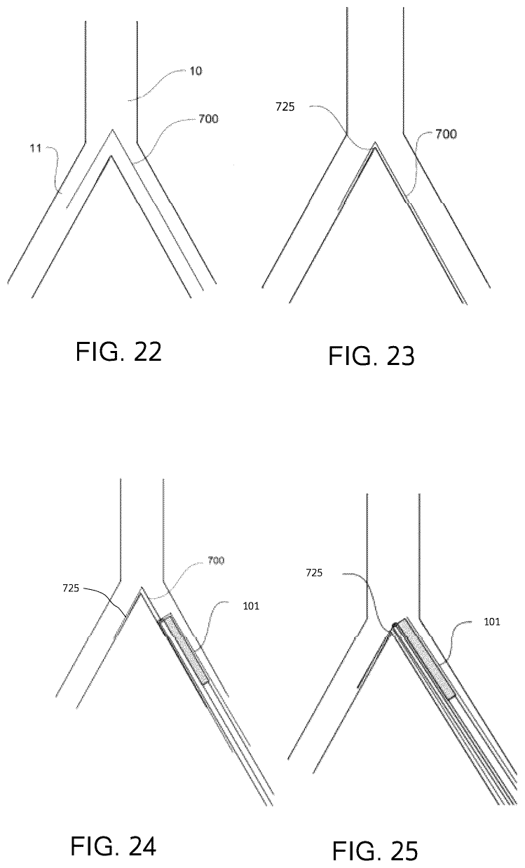

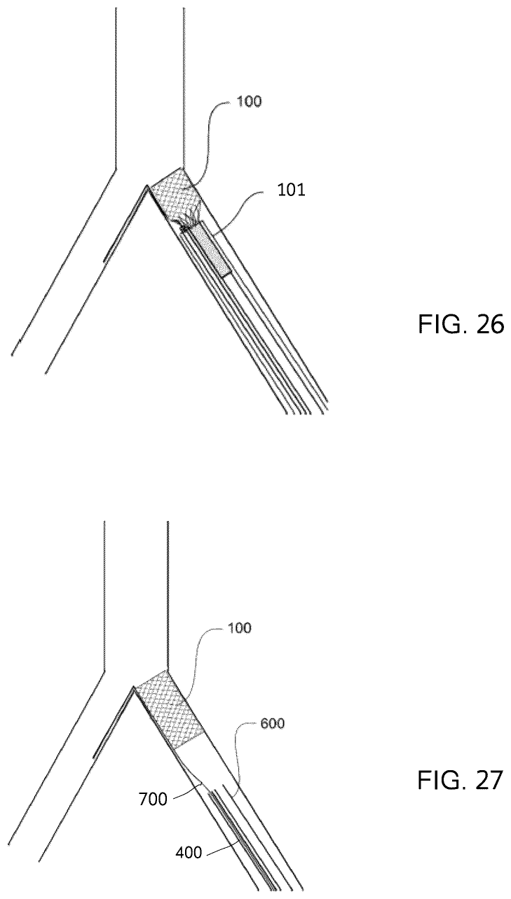

Another embodiment of a stent deployment system of the present invention is shown in FIG. 19 where the stent 101 and pusher rod 500 are combined with a wire guide catheter 400 as shown in FIG. 19B. The wire guide catheter accepts a vascular wire 700 (which can act as a form of the stent assist wire) that has a preformed distal angled section 725 shown in FIG. 19A. The fully assembled deployment system is shown in FIG. 19C. The deployment of this implementation of the present invention is illustrated in FIGS. 20 through 27. A standard vascular catheter 200 is advanced from the site of introduction into the body to the contralateral iliac vein as shown in FIG. 20. The angled wire 700 is then advanced through the catheter 200 and positioned in the iliac vein as shown in FIG. 21 and the catheter 200 is removed, leaving the angled wire 700 as shown in FIG. 22. The angled wire 700 is pulled back until it engages the medial confluence of the iliac veins and the inferior vena cava as shown in FIG. 23. The deployment system including a stent 101 is then advanced over the angled wire 700 as shown in in FIG. 24 and advanced until it abuts the apex of the wire angle positioned at the confluence of the common iliac vessels as shown in FIG. 25. The delivery system is then withdrawn as shown in FIG. 26, causing the stent 100 to partially deploy and then fully deploy as shown in FIGS. 26 and 27. The angled wire 700 is then removed and the rest of the deployment system withdrawn from the vascular system as illustrated in FIG. 27.

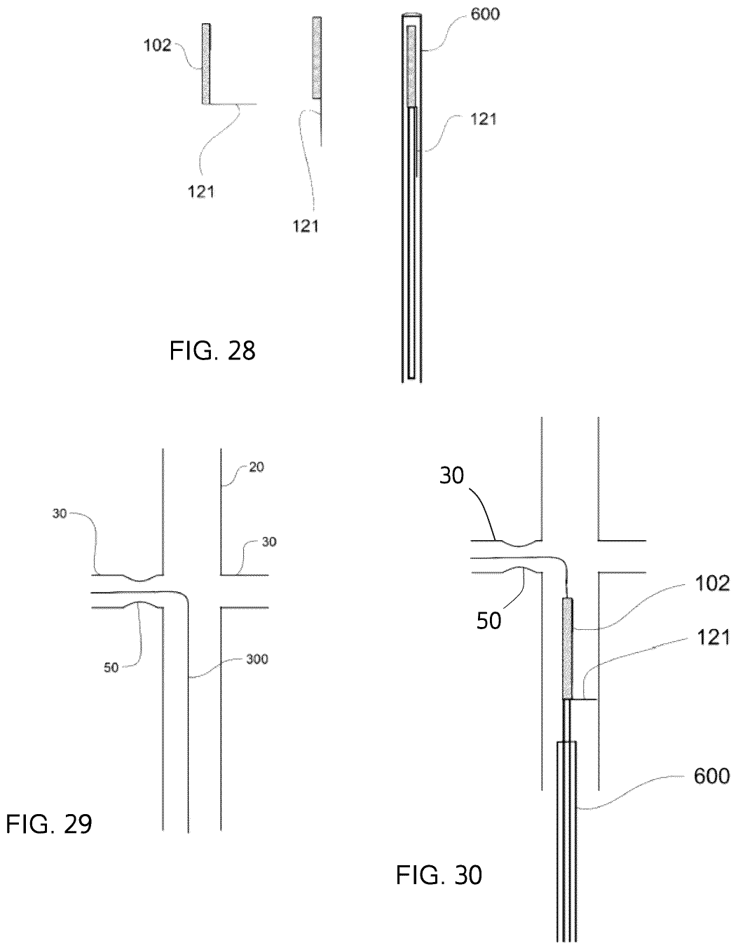

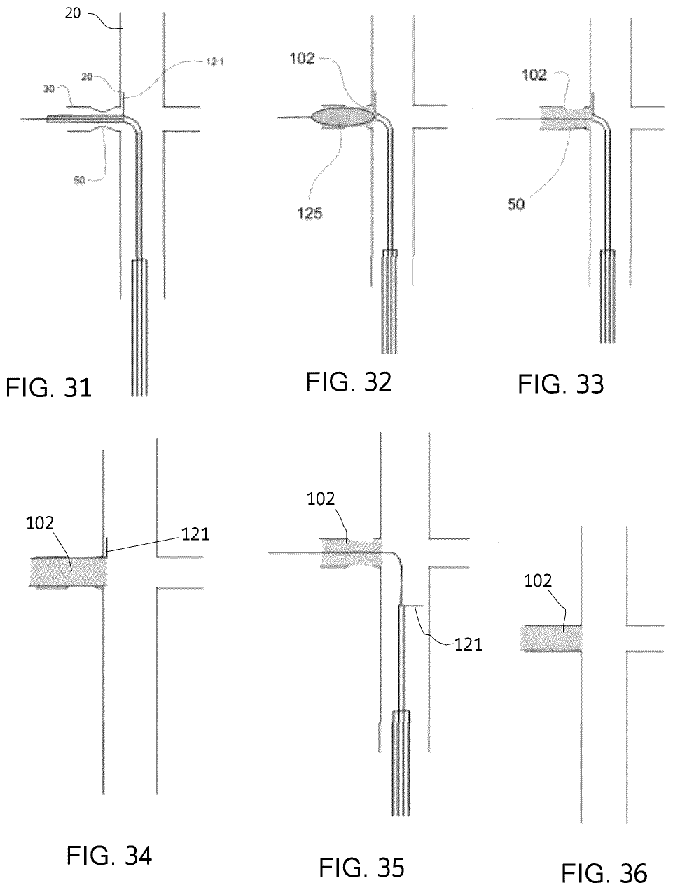

Branching vascular vessels can result in the need for a similarly accurate stent deployment system but with a configuration where the stent assist wire is positioned on the trailing end of the stent. An implementation of the present invention with a backend stent assist wire 121 is shown in FIG. 28 in its extended (left) and unextended (center) position, as well as within a deployment sheath 600 (right). FIG. 29 illustrates an example of a branching vascular structure consisting of the abdominal aorta 20 and its perpendicularly aligned branching renal arteries 30. In this example there is a stenosis 50 in the left renal artery branch. A guide wire 300 is advanced through the aorta and into this left renal artery branch as shown in FIG. 29. FIG. 30 shows the deployment system of FIG. 28 advanced over the guide wire and pulled back to reveal unexpanded stent 102 and the opened stent assist wire 121 at the end of the pusher rod. The pusher rod 500 is then advanced over the guide wire to position the stent within the narrowing 50 of the artery 30 as shown in FIG. 31. The attainment of this position is indicated to the clinician when the extended stent assist wire 121 contacts the wall of the aorta 20 as illustrated in FIG. 31.

The stent 102 is of the non self-expanding type and a balloon catheter 125 is used to expand the stent to its desired size. The deflated balloon is inserted into the stent and inflated to expand the stent to its desired size, which in this example opens the stenotic region of the renal artery as illustrated in FIG. 32. The balloon is then deflated and withdrawn, leaving the expanded stent 102 with its stent assist wire in place as shown in FIG. 33, followed by removal of the remaining deployment apparatus as illustrated in FIG. 34. As an option, the stent assist wire can be made of resorbable/dissolvable materials, as noted above.

FIGS. 35 and 36 illustrate a variation of this procedure in which the stent assist wire 121 is attached to the pusher rod rather than the stent, and is removed with withdrawal of the deployment system as shown in FIG. 35. This leaves the expanded stent 102 in place without a remaining stent assist wire as shown in FIG. 36.

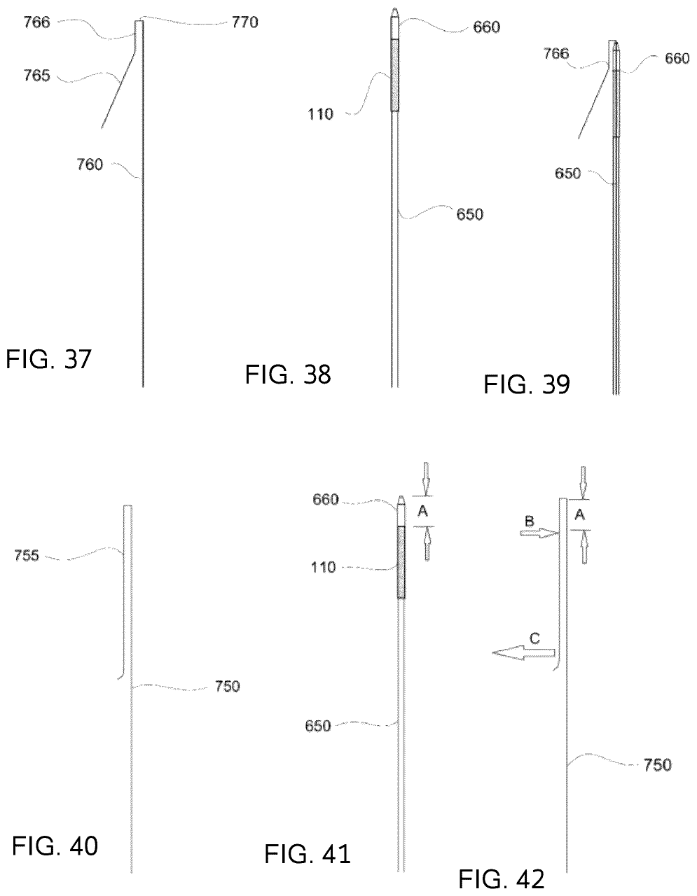

Vascular stents which are currently being marketed have various configurations of the deployment catheter tip, which are designed to retain the unexpanded stent compactly packaged and positioned for catheter deployment and subsequent implantation. The following implementations enable the delivery of these various stent configurations with the guidance of a stent assist wire of the present invention, providing precise placement of different commercially available stent packages. One such stent package design is illustrated in FIG. 38 and comprises an unexpanded stent 110 with an extended tip 660 inside a delivery catheter 650. This extended tip design requires a modification of the stent assist wire to accommodate the extra length of the stent packaging. As shown in FIG. 37 a wire 760 has a terminal blunted end 770 with a retrograde extension piece 766 which terminates in an angled stent assist wire 765. The fully assembled stent, catheter and stent assist wire is shown in FIG. 39 as it appears when ready for stent deployment.

Rather than require a clinic to stock a variety of differently shaped stent assist wire wires 760 for a variety of different stent designs, a preferred implementation starts with a common wire 750 with a bendable section 755 as shown in FIG. 40. When the clinician sees the design of the stent packaging for a given procedure, the bendable section 755 is then formed to match the dimensions of the stent packaging. FIG. 41 shows the extended tip design 660 of the stent packaging being measured and found to have a length A. An arrow B in FIG. 42 shows the point at which the bendable section is bent outward in the direction indicated by arrow C to accommodate the length A of the extended tip. The now-customized wire 750 is thus formed and dimensioned to provide stent assist wire guidance for the delivery of the stent packaging of FIG. 41. The bent configuration enables engagement into a contralateral vessel during delivery and the unbent section of length A will accommodate the extra tip length of the stent packaging. With the system in position the distal end of the stent will approximate the edge of the patient's vessel for accurate deployment.

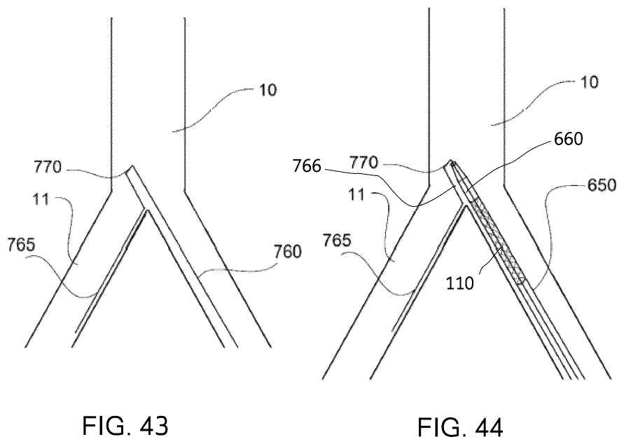

When treating a patient with the wire 760 shown in FIG. 37 or the customized wire 750 shown in FIG. 42, the fully assembled stent deployment system can be advanced to the site of the procedure, but preferably the wire 750 or 760 is advanced into the vessel first and snugged down against the medial vessel wall as shown in FIG. 43. The stent delivery catheter 650 is advanced over the wire 750 or 760 until the distal tip 660 abuts the blunted terminal end 770 of the wire as shown in FIG. 44. As this drawing clearly shows, the distal end 660 of the stent then matches the angle of the extension piece 766, with the stent assist wire 765 positioned in the contralateral vessel 11. The end of the stent 110 is position directly adjacent to the edge of the vessel for accurate deployment. The stent can then be released as previously described, and the other components of the deployment system withdrawn from the body.

Other variations of the stent deployment system of the present invention will readily occur to those skilled in the art. The stent assist wire can be attached to various components of the deployment system, for instance, including the stent delivery sheath, the stent pusher rod as shown in FIG. 35, or to other components of the deployment system.

What is claimed is:

1: Stent deployment apparatus for deploying a vascular stent to a deployment site in a blood vessel of vasculature of a body comprising:
a vascular stent;
a delivery sheath;
a pusher rod; and
a stent assist wire, located in relation to the stent, and extending outward from the vascular stent or deployment apparatus, the stent assist wire adapted to be positioned in relation to a known feature of the vasculature,
wherein the vascular stent is adapted to be deployed when the stent assist wire is positioned in relation to the known feature.

2: The stent deployment apparatus of claim 1, wherein the stent assist wire is attached to the vascular stent.

3: The stent deployment apparatus of claim 1, wherein the stent assist wire is attached to the delivery sheath.

4: The stent deployment apparatus of claim 1, wherein the stent assist wire is attached to the pusher rod.

5: The stent deployment apparatus of claim 1, wherein the stent assist wire is not attached to the stent or other stent deployment apparatus.

6: The stent deployment apparatus of claim 1, wherein the stent assist wire is adapted to be positioned at a confluence of blood vessels.

7: The stent deployment apparatus of claim 1, further comprising a guide wire, the guide wire adapted to be delivered to the deployment site prior to delivery of the stent deployment apparatus,
wherein the stent deployment apparatus is adapted to be delivered to the deployment site by means of guidance provided by the guide wire.

8: The stent deployment apparatus of claim 1, wherein the stent assist wire is attached to extend from the leading end of the vascular stent.

9: The stent deployment apparatus of claim 1, wherein the stent assist wire is attached to extend from the trailing end of the vascular stent.

10: The stent deployment apparatus of claim 1, wherein the stent assist wire further comprises a bendable wire;
wherein the bendable wire is adapted to be bent to form a section of the stent assist wire extending outward from the vascular stent; and
wherein the bendable wire is further adapted to be bent to accommodate a dimension of the vascular stent.

11: A stent assist wire, comprising in combination:
a base section;
an angled section;
said angled section connected to said base section at an apex;
said apex having an unloaded angle by which said base section diverges from said angled section and which apex orients said angled section non-parallel with said base section; and
said apex loadable by forces to change to a stored angle different from said unloaded angle and to return to said unloaded angle when such forces are absent.

12: The stent assist wire of claim 11 wherein said unloaded angle of said apex is less than about 180° between said base section and said angled section.

13: The stent assist wire of claim 12 wherein said unloaded angle of said apex is less than about 90°.

14: The stent assist wire of claim 12 wherein said unloaded angle of said apex is about 90°.

15: The stent assist wire of claim 11 wherein said base section is connected to a stent.

16: The stent assist wire of claim 15 wherein said stent is spaced from said apex by a predefined distance.

17: The stent assist wire of claim 11 wherein said base section is connected to a delivery sheath.

18: The stent assist wire of claim 11 wherein said base section is connected to a pusher rod.

19: The stent assist wire of claim 11 wherein said angled section terminates at a curved distal tip, which is curved relative to other portions of said angled section.

20: A method for placing a stent within vasculature of a patient, the method including the steps of:
placing a stent assist wire at a junction in the vasculature with the stent assist wire having a base section within a first side of the junction and an angled section in a second side of the junction spaced from the first side, and with an apex between the base section and the angled section;
positioning a stent at one of the sides of the junction in the vasculature; and
expanding the stent to engage walls of the vasculature and remain in engagement with the walls.

21: The method of claim 20 wherein said placing step and said positioning step occur together with the stent attached to the stent assist wire.

22: The method of claim 20 wherein said placing step precedes said positioning step, with the stent and the stent assist wire being separate structures.

23: The method of claim 20 wherein said positioning step results in the stent being adjacent to the apex of the stent assist wire.

24: The method of claim 20 wherein said positioning step results in the stent being spaced from the apex of the stent assist wire by a predefined distance.

25: The method of claim 20 including the further step of removing the stent assist wire after said expanding step.
